(19) United States
(12) Patent Application Publication
(10) Pub. No.: US 2014/0179677 A1
(43) Pub. Date: Jun. 26, 2014

(54) THERAPEUTIC AGENT FOR ECTOPIC PREGNANCY

(71) Applicant: Kazuhiro Kawamura, Akita-shi (JP)

(72) Inventor: Kazuhiro Kawamura, Akita-shi (JP)

(73) Assignee: AKITA UNIVERSITY, Akita-shi, Akita (JP)

(21) Appl. No.: 13/977,434

(22) PCT Filed: Dec. 28, 2011
(86) PCT No.: PCT/JP2011/080466

§ 371 Date: Feb. 10, 2014

(30) Foreign Application Priority Data
Dec. 29, 2010 (JP) 2010-294391

(51) Int. Cl. C07D 498/22; G01N 33/50

(52) U.S. Cl. CPC C07D 498/22; G01N 33/5044
USPC 514/211.08; 540/545; 435/15

(57) ABSTRACT

A novel therapeutic agent for ectopic pregnancy having a therapeutic effect for ectopic pregnancy, especially unruptured ectopic pregnancy, and a novel method of screening a therapeutic agent for ectopic pregnancy are disclosed. The therapeutic agent for ectopic pregnancy contains as an effective ingredient a suppressor of brain-derived neurotrophic factor (BDNF) and/or of brain-derived neurotrophic factor receptor (TrkB). The method of screening a therapeutic agent for ectopic pregnancy includes measuring the kinase activity of TrkB in the presence of a test substance and the kinase activity of TrkB in the absence of the test substance; and selecting the test substance which decreases the kinase activity of TrkB.

### TECHNICAL FIELD

The present invention relates to a therapeutic agent for ectopic pregnancy.

### BACKGROUND ART

Ectopic pregnancy is a life-threatening condition in the first trimester of gestation (Non-patent Document 1). Recent advances in serial hormone assays and transvaginal ultrasonography facilitates the diagnosis and treatment of ectopic pregnancy before rupture. Early diagnosis and timely treatment have resulted in a dramatic decline in mortality because of ectopic pregnancy (Non-patent Document 1). Until the mid 1980s, treatment for ectopic pregnancy was exclusively surgical. In 1982, the present inventors reported treatment of an interstitial ectopic pregnancy in a patient with a 15-day course of intramuscular methotrexate (MTX) (Non-patent Document 2). Subsequently, MTX treatment has been accepted as a medical treatment for unruptured ectopic pregnancy. MTX is a folic acid antagonist that interferes with DNA synthesis and thus highly toxic to rapidly replicating tissues and malignant cells. However, signs of advanced ectopic pregnancy, such as detection of embryonic cardiac activity, high human chorionic gonadotropin (hCG) level, and large (>4 cm) size of conceptus are contraindications to MTX treatment (Non-patent Document 3). Furthermore, gastric distress, nausea, vomiting, stomatitis, canker sore, and dizziness are commonly observed as side effects of MTX treatment (Non-patent Document 3). Thus, development of more potent and safer medical treatment is needed. Human villous trophoblast is composed of cytotrophoblast and syncytiotrophoblast layers. Cytotrophoblasts display highly proliferative and invasive properties in the first trimester of gestation, whereas syncytiotrophoblasts are differentiated following fusion of cytotrophoblasts and have little potential for proliferation throughout pregnancy. Cytotrophoblasts also differentiate into highly invasive cells, called extravillous trophoblasts (EVTs), that break out of the chorionic villi, migrate into maternal decidua, and invade myometrium, leading to a remodeling of the utero-placental arteries for adequate supply of maternal blood necessary for fetal growth. Proliferation of trophoblasts in placental villi is observed in the cytotrophoblasts as well as in the EVTs before they migrate out of the villi (Non-patent Document 4, Non-patent Document 5).

Brain-derived neurotrophic factor (BDNF) is a member of the neurotrophin family of proteins known to activate the high affinity tyrosine kinase B (TrkB) receptor together with the pan-neurotrophin low-affinity co-receptor p75 (p75NTR) (Non-patent Document 6). Following BDNF binding, TrkB receptor plays important roles in cell differentiation, proliferation and survival in different cell types (Non-patent Document 6, Non-patent Document 7). Although neurotrophins are widely expressed in the central nervous system and are important for neuronal differentiation and survival (Non-patent Document 8), they also play important roles in nonneuronal tissues (Non-patent Document 9). The present inventors recently found the expression of TrkB and its ligands, BDNF and neurotrophin-4/5 (NT-4/5) in trophectoderm cells of blastocyst stage embryos capable of differentiating into invasive trophoblasts, and demonstrated promotional effects of BDNF on the proliferation and survival of trophectoderm cells before implantation (Non-patent Document 10). After implantation, the expression of TrkB and its ligands persists in placental trophoblast cells, and the present inventors demonstrated autocrine/paracrine regulatory roles of the TrkB signaling system in trophoblast cell growth and survival during placental development in mice (Non-patent Document 11). In human, the present inventors further showed important autocrine roles of the BDNF/TrkB signaling system in malignant trophoblastic, choriocarcinoma cell growth (Non-patent Document 12).

### PRIOR ART DOCUMENTS

### Non-Patent Documents

Non-patent Document 1: Berg C J, Chang J, Callaghan W M, Whitehead S J 2003 Pregnancy-related mortality in the United States, 1991-1997. Obstet Gynecol 101:289-296
Non-patent Document 2: Tanaka T, Hayashi H, Kutsuzawa T, Fujimoto S, Ichinoe K 1982 Treatment of interstitial ectopic pregnancy with methotrexate: report of a successful case. Fertil Steril 37:851-852
Non-patent Document 3: American Society for Reproductive Medicine 2008 Medical treatment of ectopic pregnancy. Fertil Steril 90:S206-212
Non-patent Document 4: Pijnenborg R, Bland J M, Robertson W B, Brosens I 1983 Uteroplacental arterial changes related to interstitial trophoblast migration in early human pregnancy. Placenta 4:397-413
Non-patent Document 5: Aplin J D 1991 Implantation, trophoblast differentiation and haemochorial placentation: mechanistic evidence in vivo and in vitro. J Cell Sci 99:681-692
Non-patent Document 6: Barbacid M 1994 The Trk family of neurotrophin receptors. J Neurobiol 25:1386-1403
Non-patent Document 7: Huang E J, Reichardt L F 2003 Trk receptors: roles in neuronal signal transduction. Annu Rev Biochem 72:609-642
Non-patent Document 8: Jones K R, Farinas I, Backus C, Reichardt L F 1994 Targeted disruption of the BDNF gene perturbs brain and sensory neuron development but not motor neuron development. Cell 76:989-999
Non-patent Document 9: Ip N Y, Stitt T N, Tapley P, Klein R, Glass D J, Fandl J, Greene L A, Barbacid M, Yancopoulos G D 1993 Similarities and differences in the way neurotrophins interact with the Trk receptors in neuronal and nonneuronal cells. Neuron 10:137-149
Non-patent Document 10: Kawamura K, Kawamura N, Fukuda J, Kumagai J, Hsueh A J, Tanaka T 2007 Regulation of preimplantation embryo development by brain-derived neurotrophic factor. Dev Biol 311:147-158
Non-patent Document 11: Kawamura K, Kawamura N, Sato W, Fukuda J, Kumagai J, Tanaka T 2009 Brain-derived neurotrophic factor promotes implantation and subsequent placental development by stimulating trophoblast cell growth and survival. Endocrinology 150:3774-3782
Non-patent Document 12: Kawamura N, Kawamura K, Manabe M, Tanaka T 2010 Inhibition of Brain-Derived Neurotrophic Factor/Tyrosine Kinase B Signaling Suppresses Choriocarcinoma Cell Growth. Endocrinology 151:3006-3014
Non-patent Document 13: Genbacev O, Schubach S A, Miller R K 1992 Villous culture of first trimester human placenta—model to study extravillous trophoblast (EVT) differentiation. Placenta 13:439-461
Non-patent Document 14: Tapley P, Lamballe F, Barbacid 1 M 1992 K252a is a selective inhibitor of the tyrosine protein 2 kinase activity of the trk family of oncogenes and neurotrophin receptors. Oncogene 7:371-381
Non-patent Document 15: Ross A H, McKinnon C A, Daou M C, Ratliff K, Wolf D E 1995 Differential biological effects of K252 kinase inhibitors are related to membrane solubility but not to permeability. J Neurochem 65:2748-2756
Non-patent Document 16: Liu D, Li C, Chen Y, Burnett C, Liu X Y, Downs S, Collins R D, Hawiger J 2004 Nuclear import of proinflammatory transcription factors is required for massive liver apoptosis induced by bacterial lipopolysaccharide. J Biol Chem 279:48434-48442
Non-patent Document 17: Kawamura K, Fukuda J, Shimizu Y, Kodama H, Tanaka T 2005 Survivin contributes to the anti-apoptotic activities of transforming growth factor alpha in mouse blastocysts through phosphatidylinositol 3′-kinase pathway. Biol Reprod 73:1094-1101
Non-patent Document 18: Red-Horse K, Rivera J, Schanz A, Zhou Y, Winn V, Kapidzic M, Maltepe E, Okazaki K, Kochman R, Vo K C, Giudice L, Erlebacher A, McCune J M, Stoddart C A, Fisher S J 2006 Cytotrophoblast induction of arterial apoptosis and lymphangiogenesis in an in vivo model of human placentation. J Clin Invest 116:2643-2652
Non-patent Document 19: Nakaigawa N, Yao M, Baba M, Kato S, Kishida T, Hattori K, Nagashima Y, Kubota Y 2006 Inactivation of von Hippel-Lindau gene induces constitutive phosphorylation of MET protein in clear cell renal carcinoma. Cancer Res 66:3699-3705
Non-patent Document 20: Zuckermann F A, Head J R 1986 Isolation and characterization of trophoblast from murine placenta. Placenta 7:349-364
Non-patent Document 21: Le Bouteiller P, Solier C, Proll J, Aguerre-Girr M, Fournel S, Lenfant F 1999 Placental HLA-G protein expression in vivo:where and what for? Hum Reprod Update 5:223-233
Non-patent Document 22: Bischof P, Meisser A, Campana A 2000 Paracrine and autocrine regulators of trophoblast invasion—a review. Placenta 21 Suppl A:S55-60
Non-patent Document 23: Koide Y, Aoki T, Hreshchyshyn M M 1971 Effects of hormones, methotrexate, and dactinomycin on benign trophoblast. Am J Obstet Gynecol 109:453-456
Non-patent Document 24: James J L, Stone P R, Chamley L W 2006 The regulation of trophoblast differentiation by oxygen in the first trimester of pregnancy. Hum Reprod Update 12:137-144
Non-patent Document 25: Caniggia I, Mostachfi H, Winter J, Gassmann M, Lye S J, Kuliszewski M, Post M 2000 Hypoxia-inducible factor-1 mediates the biological effects of oxygen on human trophoblast differentiation through TGFbeta(3). J Clin Invest 105:577-587
Non-patent Document 26: Semenza G L 2003 Targeting HIF-1 for cancer therapy. Nat Rev Cancer 3:721-732
Non-patent Document 27: Shi Q, Zhang P, Zhang J, Chen X, Lu H, Tian Y, Parker T L, Liu Y 2009 Adenovirus-mediated brain-derived neurotrophic 1 factor expression regulated by hypoxia response element protects brain from injury of transient middle cerebral artery occlusion in mice. Neurosci Lett 465:220-225
Non-patent Document 28: Martens L K, Kirschner K M, Warnecke C, Scholz H 2007 Hypoxia-inducible factor-1 (HIF-1) is a transcriptional activator of the TrkB neurotrophin receptor gene. J Biol Chem 282:14379-14388
Non-patent Document 29: Handschuh K, Guibourdenche J, Tsatsaris V, Guesnon M, Laurendeau I, Evain-Brion D, Fournier T 2007 Human chorionic gonadotropin produced by the invasive trophoblast but not the villous trophoblast promotes cell invasion and is down-regulated by peroxisome proliferator-activated receptor-gamma. Endocrinology 148:5011-5019
Non-patent Document 30: Watson A L, Palmer M E, Burton G 1995 Human chorionic gonadotrophin release and tissue viability in placental organ culture. Hum Reprod 10:2159-2164
Non-patent Document 31: Kar M, Ghosh D, Sengupta J 2007 Histochemical and morphological examination of proliferation and apoptosis in human first trimester villous trophoblast. Hum Reprod 22:2814-2823
Non-patent Document 32: Kawamura K, Kawamura N, Mulders S M, Sollewijn Gelpke M D, Hsueh A J 2005 Ovarian brain-derived neurotrophic factor (BDNF) promotes the development of oocytes into preimplantation embryos. Proc Natl Acad Sci USA 102:9206-9211
Non-patent Document 33: Klein R, Conway D, Parada L F, Barbacid M 1990 The trkB tyrosine protein kinase gene codes for a second neurogenic receptor that lacks the catalytic kinase domain. Cell 61:647-656
Non-patent Document 34: Wang T et al., 2008, Identification of 4-aminopyrazolylpyrimidines as potent inhibitors of Trk kinases, J. Med. Chem, 12; 51(15):4672-84
Non-patent Document 35: Somaiah N and Simon G R, 2009, Molecular targeted therapy in non-small cell lung cancer: an overview of available agents, J. Thorac. Oncol., 4, S 1045-83
Non-patent Document 36: Michael D. Sadick et al., 1997, Analysis of Neurotrophin/Receptor Interactions with a gD-Flag-Modified Quantitative Kinase Receptor Activation (gD.KIRA) Enzyme-Linked Immunosorbent Assay, Experimental Cell Research, 234, 354-361
Non-patent Document 37: Anderson R A et al., 2010, Brain-derived neurotrophic factor is a regulator of human oocyte maturation and early embryo development, Fertil Steril, 15, 93(5), 1394-406
Non-patent Document 38: Nanami KAWAMURA et al., Program and Abstract of Meeting of the Japan Trophoblastic Diseases Society•Japan Placenta Association, Vol. 28th-18th Page. 38 (2010), choriocarcinoma cell growth action and its molecular mechanism of Brain-derived neurotrophic factor (BDNF)/tyrosine kinase B (trkB) signal
Non-patent Document 39: Kazuhiro KAWAMURA et al., Program and Abstract of Meeting of the Japan Trophoblastic Diseases Society•Japan Placenta Association: Vol. 28th-18th Page. 65 (2010), Promotion of implantation and placental development by brain-derived neurotrophic factor (BDNF) and analysis its molecular mechanism
Non-patent Document 40: Kazuhiro KAWAMURA et al., Journal of Japan Society for Reproductive Medicine, Vol. 54 No. 4 Page. 324 (2009), Identification of novel factor controlling implantation and plancental development and clarification of its molecular mechanism: brain-derived neurotrophic factor (BDNF)
Non-patent Document 41: Kazuhiro KAWAMURA et al., Acta Obstettrica et Gynaecologica Japonica, Vol. 61 No. 10 Page. 1935-1944 (2009), Influence by central nerve-related physiologically active substance on oocyte maturation, embryonic development and implantation
Non-patent Document 42: Kazuhiro KAWAMURA et al., Folia Endocrinologica Japonica, Vol. 85 No. 2 Page. 657 (2009), Control of implantation and plancental development by brain-derived neurotrophic factor (BDNF) and its molecular mechanism

### SUMMARY OF THE INVENTION

### Problems to be Solved by the Invention

An object of the present invention is to provide a novel therapeutic agent for ectopic pregnancy having a therapeutic effect for ectopic pregnancy, especially unruptured ectopic pregnancy. Another object of the present invention is to provide a novel method of screening a therapeutic agent for ectopic pregnancy.

### Means for Solving the Problems

The present inventors intensively studied to discover that growth of cytotrophoblast cells can be suppressed by suppressing the action of BDNF and/or TrkB, and treatment of ectopic pregnancy can be attained thereby, to complete the present invention.

That is, the present invention provides a therapeutic agent for ectopic pregnancy, comprising as an effective ingredient a suppressor of brain-derived neurotrophic factor (BDNF) and/or of brain-derived neurotrophic factor receptor (TrkB). The present invention also provides a method of screening a therapeutic agent for ectopic pregnancy, the method comprising measuring the kinase activity of TrkB in the presence of a test substance and the kinase activity of TrkB in the absence of the test substance; and selecting a test substance which decreases the kinase activity of TrkB. The present invention further provides a method of screening a therapeutic agent for ectopic pregnancy, the method comprising the following steps (a) to (d):
(a) preparing model animals in which human placental villi are transplanted to a renal tissue of a mammal other than human;
(b) administering a test sample to one (or one population) of the model animals prepared and raising the animal(s), and administering only the carrier in the test sample to another (or another population) of the model animals prepared and raisin the animal(s);
(c) comparing cytotrophoblast cells and extravillous trophoblast cells in the renal tissue in the model animal(s) to which the test sample was administered with cytotrophoblast cells and extravillous trophoblast cells in the renal tissue in the model animal(s) to which the test sample was not administered; and
(d) selecting the test sample as a therapeutic agent for ectopic pregnancy, which test sample decreased cytotrophoblast cells and extravillous trophoblast cells in the renal tissue in the model animal(s) to which the test sample was administered.

The present invention still further provides a suppressor of brain-derived neurotrophic factor (BDNF) and/or of brain-derived neurotrophic factor receptor (TrkB) for use in the treatment of ectopic pregnancy.

The present invention still further provides a method of treating ectopic pregnancy, the method comprising administering an effective amount of a suppressor of brain-derived neurotrophic factor (BDNF) and/or of brain-derived neurotrophic factor receptor (TrkB) to a patient with ectopic pregnancy.

### Effects of the Invention

By the present invention, a novel therapeutic agent for ectopic pregnancy having an excellent therapeutic effect for ectopic pregnancy and a screening method thereof were provided.

### BRIEF DESCRIPTION OF THE DRAWINGS

FIG. 1 shows the temporal and spatial expression of BDNF, NT-4/5, and TrkB in human placental villi of intrauterine and ectopic pregnancy, observed in the Example below. (A) Temporal expression of BDNF, NT-4/5, and TrkB in human placental villi during first trimester of gestation. BDNF and NT-4/5 protein or TrkB transcript levels were quantified using ELISA (BDNF and NT-4/5) or real-time RT-PCR (TrkB), respectively. Levels of BDNF and NT-4/5 proteins and TrkB mRNA were detected using samples obtained at different pregnant weeks (n=4-6 donors). Levels of TrkB mRNA were normalized using transcript levels of β-actin in the same sample. Columns, mean; bars, SE. *, P<0.05 vs. 6 weeks of pregnancy. Immunohistochemical detection of BDNF and TrkB in human placental villi obtained from donors (B) and diseased tissues from a patient with ectopic pregnancy (C), both at 8 weeks of pregnancy. In placental villi, BDNF was found in syncytiotrophoblasts (arrows) and extravillous trophoblasts (EVTs). In contrast, TrkB was found in cytotrophoblasts (arrowheads) and extravillous trophoblasts. Upper and middle panels are specific staining, whereas lower panels depict sections stained with nonimmune IgG and serve as controls. Insert: higher magnification of the image indicated in original figure. (Scale bars, 100 μm.).

FIG. 2 shows the effects of in vitro suppression of endogenous TrkB signaling on human trophoblast differentiation, observed in the Example below. Villous explants at 6-8 weeks of gestation were cultured in medium alone (control, C), with different doses of TrkB ectodomain (TrkB EC), with K252a, or the plasma membrane nonpermeable K252b under 3% O₂. (A) Morphological changes in villous explants at 48 and 96 h of culture. Representative images were obtained from villous explants treated with or without TrkB ectodomain (10 μg/ml), K252a (1,000 nM) or K252b (1,000 nM). Outgrowth of EVTs was found in the distal end of the villous tips and inhibited following treatment with either TrkB EC or K252a. (Scale bars, 100 μm.). Inhibition of EVT outgrowth (B) and HLA-G transcript levels (C) following suppression of endogenous TrkB signaling at 96 h of culture. EVT outgrowth was quantified based on the proportions of EVT outgrowth. EVT positive was defined as ≧50% anchoring villous tips showed cell outgrowth (n=12-13). Transcript levels for HLA-G were determined using real-time RT-PCR. Data were expressed as fold decreases relative to controls and normalized to 1.0. Columns, mean; bars, SE. *, P<0.05 vs. control group.

FIG. 3 shows the effects of in vitro suppression of endogenous TrkB signaling on human trophoblast viability. Villous explants at 6-8 weeks of gestation were cultured in medium alone (control, C), with TrkB ectodomain (10 μg/ml), K252a (1,000 nM) or K252b (1,000 nM) under 3% O₂ for 96 h. (A) Histological characterization of cell proliferation in cultured villous explants using H&E staining (upper), and immunodetection of PCNA (middle) and Ki-67 (lower). H&E staining shows decreases in number of villous cytotrophoblasts (arrowheads 1), but not of syncytiotrophoblasts (arrows), and partial detachment of trophoblast layers (arrowheads 2) following either TrkB EC or K252a treatment. Both PCNA and Ki-67 signals (brown) were decreased in remaining cytotrophoblasts (arrowheads 3) following treatment with different inhibitors. Inserts: higher magnification of selected areas; M: matrigel. (Scale bars, 100 μm.). (B) Decreases in glucose utilization of villous explants during culture following treatment with either TrkB EC or K252a. Media were changed at day 2 of culture and samples were obtained after 48 h of culture (n=4). Glucose concentrations in media were quantified using an enzymatic assay. Columns, mean; bars, SE. *, P<0.05 vs. control group.

FIG. 4 shows the effects of in vitro suppression of endogenous TrkB signaling on human trophoblast survival, observed in the Example below. Villous explants at 6-8 weeks of gestation were cultured in medium alone (control, C), with TrkB ectodomain (10 μg/ml), K252a (1,000 nM) or K252b (1,000 nM) under 3% O₂ for 96 h. (A) Detection of DNA fragmentation in cultured villous explants using in situ TUNEL staining. Cellular nucleic acids were stained using propidium iodide (red signals). The numbers of positive apoptosis signals (green fluorescence) were increased in the cytotrophoblasts (arrowheads) following TrkB ectodomain or K252a treatment. (Scale bars, 100 μm.). Inserts: higher magnification selected areas; arrows: syncytiotrophoblasts. (B) Increases in caspase-3/7 activities in cultured villous explants following treatment with either TrkB EC or K252a. Data were expressed as fold increases relative to controls and normalized to 1 (n=4). Columns, mean; bars, SE. *, P<0.05 vs. control group.

FIG. 5 shows the xenotransplantation of human villi into SCID mice as an in vivo model of ectopic pregnancy, observed in the Example below. Villous grafts at 7-8 weeks of gestation were surgically placed under the kidney capsule of SCID mice and maintained for 1-3 weeks before histological (A) and biochemical (B) analyses. (A) Histological evaluation of human villi growth in the mouse kidneys during 3 weeks after xenotransplantation. Human trophoblasts were detected by cytokeratin immunohistochemistry. At 1 week after xenotransplantation, human trophoblasts invaded into renal tissue of mouse kidney (arrows) from original transplantation sites marked by villous cores (arrowheads). At 3 weeks, the areas of mouse kidney occupied by human trophoblasts were expanded and trophoblast invasion was extended to deeper regions of the kidney. (Scale bars, 400 μm.). (B) Changes of hCG-β levels in tissue homogenates of grafted kidneys during 3 weeks of xenotransplantation. Tissue hCG-β levels were quantified using RIA (n=6-15). Points, mean; bars, SE. (C) Identification of EVTs by immunodetection of HLA-G in the kidney at 2 weeks after xenotransplantation of human villi. HLA-G was found in trophoblasts invading into kidney (arrowheads), while HLA-G was absent in other cell types of trophoblasts stained with cytokeratin. (Scale bars, 200 μm.).

FIG. 6 shows the suppression of endogenous TrkB signaling led to in in vivo growth inhibition of human trophoblasts in a model of ectopic pregnancy, observed in the Example below. SCID mice at 1 week after xenotransplantation of human villi (7-8 weeks of gestation) under the kidney capsule were treated without (vehicle) or with K252a (500 μg/kg), K252b (500 μg/kg), or MTX (1 mg/kg) daily for 7 days. (A-C) Histological characterization of trophoblast cell proliferation and apoptosis in transplanted villi. Representative images were obtained from resected kidneys at 8 days after treatment. Cytokeratin (A, upper panels), HLA-G (A, lower panels), and H&E staining (B, upper panels), showed decreased numbers of invading EVTs and cytotrophoblasts following K252a treatment. (Scale bars, A: 400 μm; B: 100 μm.). Cell proliferation was detected using PCNA (B, middle panels) and Ki-67 (B, lower panels) immunostaining, whereas apoptosis was estimated using in situ TUNEL staining (C). PCNA and Ki-67 signals (brown) decreased, whereas TUNEL stained nuclei (green fluorescence) increased in cytotrophoblasts following K252a treatment. (Scale bars, 100 μm.). (D-F) Decreased HLA-G transcript levels (D) and hCG-β protein levels (E) as well as increased caspase-3/7 activities (F) found in renal homogenates with transplanted villi following treatment with K252a. Samples were obtained from the mice at 8 days after treatment (n=10-15). Transcript levels of HLA-G and caspase-3/7 activities were expressed as fold increases relative to controls (vehicle alone) and normalized to 1. Columns, mean; bars, SE. *, P<0.05 vs. control group.

FIG. 7 shows the lack of cell proliferation activity in the migrating EVTs of villous explants, observed in the Example below. Representative images were obtained from villous explants at 6-8 weeks of gestation cultured under 3% O₂. Cell proliferation activity in migrating EVTs was determined by immunodetection of Ki-67 and HLA-G, and H&E staining. EVTs were negative for Ki-67, a marker for cell proliferation, but positive for HLA-G, a specific marker for EVTs (arrows).

FIG. 8 shows the expression of BDNF, and TrkB in human placental villi and mouse renal tissues, observed in the Example below. BDNF and TrkB transcript levels were quantified using real-time RT-PCR. Levels of BDNF and TrkB mRNA were detected using human villous samples obtained at 8 weeks of gestation or mouse renal samples dissected from the kidney (n=4-6 donors or 4 animals). Levels of TrkB mRNA were normalized using transcript levels of β-actin in the same sample. Columns, mean; bars, SE. N.D.: not detected.

FIG. 9 shows the expression of Trk ligands (NGF and NT-3) and receptors (TrkA and TrkC), and truncated TrkB in human placental villi during first trimester of gestation, observed in the Example below. Expression of Trk ligands and receptors mRNAs in the placental villi was detected by RT-PCR. Levels of β-actin serve as loading controls. No template DNA was included for negative controls (NC).

### MODE FOR CARRYING OUT THE INVENTION

As described above, the therapeutic agent for ectopic pregnancy of the present 2 0 invention contains as an effective ingredient(s) a suppressor of BDNF and/or TrkB. The term “suppressor of BDNF and/or TrkB” herein means (1) a substance which suppresses the physiological action of at least one of BDNF and TrkB; (2) a substance which suppresses the biding between BDNF and TrkB, or (3) a substance which suppresses the production in a cell of at least one of BDNF and TrkB. Examples of the substance (1) include tyrosine kinase inhibitors. Examples of the substance (2) include (i) free TrkB and TrkB fragments having an ability to bind to BDNF, and (ii) antibodies to TrkB or BDNF. Examples of the substance (3) include (i) interfering RNAs against BDNF gene or TrkB gene, and vectors producing such interfering RNAs in a cell, and (ii) antisense nucleic acids against BDNF gene or TrkB gene and recombinant vectors producing such antisense nucleic acids in a cell. These are now hereinbelow described.

TrkB has a tyrosine kinase activity. As concretely shown in the Examples below, growth of cytotrophoblast cells can be suppressed by inhibiting the tyrosine kinase activity so that the therapeutic effect against ectopic pregnancy is exerted. Therefore, a tyrosine kinase inhibitor (suppressor) can be used as the effective ingredient of the therapeutic agent for ectopic pregnancy of the present invention. Various tyrosine kinase inhibitors are known and not a few of them are commercially available. Commercially available tyrosine kinase inhibitors can preferably be employed. Examples of the known tyrosine kinase inhibitors include, but are not limited to, K252a, AZ-23 (Wang et al. J Med Chem 2008, 51, 4672-84; Non-patent Document 34), CEP-701 (Cephalon Inc., West Chester, Pa.), CEP-751 (Kyowa Hakko Kogyo, Tokyo, Japan), CEP-2563 (Cephalon Inc.) and CEP-7801 (Somaiah et al. J Thorac Oncol, 2009,4, S1045-83; Non-patent Document 35).

As the tyrosine kinase inhibitor, the compounds represented by Formula (1) or (2) below whose tyrosine kinase inhibitory activities have been demonstrated in JP 3,344,586 (Patent Document 1) can also be used.

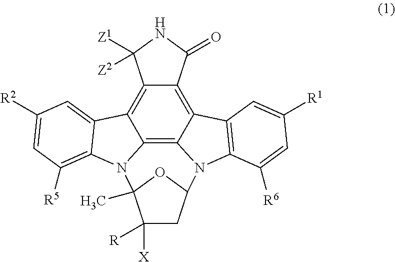

(wherein
a) both of Z¹ and Z² are hydrogen;
1) R is selected from the group consisting of OH, C₁-C₆ O-n-alkyl and C₂-C₆ O-acyl;
2) X is selected from the following group consisting of:
H;
CONHC₆H₅ with the proviso that in this case, R¹ and R² are not simultaneously Br;
CH₂ Y wherein Y is OR⁷ (wherein R⁷ is H or C₂-C₅ acyl);
SOR⁸ wherein R⁸ is C₁-C₃ alkyl, aryl or a nitrogen-containing heterocyclic group;
NR⁹R¹⁰ wherein R⁹ and R¹⁰ are independently H or C₁-C₃ alkyl, Pro, Ser, Gly, Lys or C₂-C₅ acyl with the proviso that only one of R⁹ and R¹⁰ is Pro, Ser, Gly, Lys or acyl;
SR¹⁶ wherein R¹⁶ is aryl, C₁-C₃ alkyl or a nitrogen-containing heterocyclic group;
N₃;
CO₂CH₃;
S-Glc;
CONR¹¹R¹² wherein R¹¹ and R¹² are independently H, C₁-C₆ alkyl, C₆H₅ or C₁-C₆ hydroxyalkyl, or R¹¹ and R¹² together form —CH₂CH₂OCH₂CH₂—;
CH═NNHCONH₂;
CONHOH;
CH═NOH;
CH═NNHC(═NH)NH₂;

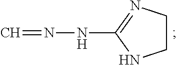

CH═NN(R¹⁷)₂ wherein R¹⁷ is aryl;
CH₂NHCONHR¹⁸ wherein R¹⁸ is lower alkyl or aryl; or
X and R together form —CH₂NHCO₂—, CH₂OH(CH₃)₂O—, ═O or —CH₂N(CH₃)CO₂;
3) R¹¹, R², R⁵ and R⁶ are independently H, or two or less of these are F, Cl, Br, I, NO₂, CN, OH, NHCONHR¹³, CH₂OR¹³, C₁-C₃ alkyl, CH₂OCONHR¹⁴ or NHCO₂R¹⁴, wherein R¹⁴ is lower alkyl; CH(SC₆H₅)₂ or CH(—SCH₂CH₂S—);
R¹ is CH₂S(O)ₚR²¹ and R², R⁵ and R⁶ are H wherein p is 0 or 1, R²¹ is aryl, C₁-C₃ alkyl, or a nitrogen-containing heterocyclic group,

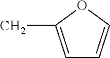

or CH₂CH₂N(CH₃)₂;
R¹ is CH═NHR²²R²³ and R², R⁵ and R⁶ are H, wherein R²² and R²³ are independently H, C₁-C₃ alkyl, C(═NH)NH₂ or a nitrogen-containing heterocyclic group, or R²² and R²³ together form —(CH₂)₄—, —(CH₂CH₂OCH₂CH₂)—or —CH₂CH₂N(CH₃)CH₂CH₂—, with the proviso that R²² and R²³ cannot be simultaneously H, and that at least one of R²² and R²³ are H except for the cases where both of these are alkyl;
(b) in cases where Z¹ and Z² together represent O, X is CO₂CH₃, R is OH, and each of R¹, R², R⁵ and R⁶ represents hydrogen).
The term “lower” herein means C₁-C₆.

The tyrosine kinase inhibitor represented by Formula (2) is shown below.

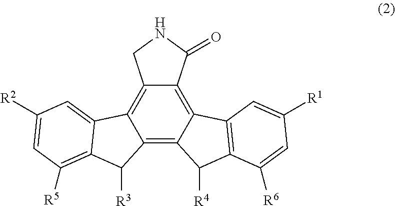

(wherein
R³ and R⁴ are independently selected from the group consisting of H, C₁-C₆ alkyl, C₁-C₃ hydroxyalkyl and C₃-C₆ alkenyl, with the proviso that R³ and R⁴ are not simultaneously H;
1) both of Z¹ and Z² are hydrogen,
R¹, R², R⁵ and R⁶ are independently H, or two or less of these are F, Cl, Br, I, NO₂, CN, OH, NHCONHR¹³, wherein R¹³ is C₆H₅ or C₁-C₃ alkyl, with the proviso that only one of R¹, R², R⁵ and R⁶ is NHCONHR¹³; CH₂OR¹³; C₁-C₃ alkyl;
CH₂OCONHC₂H₅; or NHCO₂CH₃;
2) in cases where Z¹ and Z² together represent O, each of R¹, R², R⁵ and R⁶ is hydrogen).

Among these compounds, K252a employed in the Examples below is a substance produced by a soil fungus and is widely used as a tyrosine kinase inhibitor. Since this compound is commercially available, the commercially available product can be conveniently used.

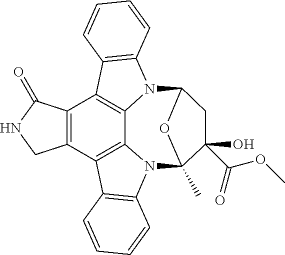 [SMILES: COC(=O)[C@@]1(O)C[C@H]2O[C@]1(C)n1c3ccccc3c3c4c(c5c6ccccc6n2c5c31)C(=O)NC4]

When a tyrosine kinase inhibitor is used as the therapeutic agent for ectopic pregnancy of the present invention, the administration route may be oral route or a parenteral route. In case of a parenteral route, it can be administered via various usual administration routes such as direct administration to the site of the ectopic pregnancy, intravenous, intramuscular, subcutaneous, intracutaneous, percutaneous, rectal and instillation routes. The dose of administration is appropriately selected depending on the type of the tyrosine kinase inhibitor, state of the patient and so on, the dose per adult per day is usually about 1 mg to 100,000 mg, preferably 1 mg to 1000 mg. However, needless to say, the dose is not restricted to this range.

In cases where a tyrosine kinase inhibitor is used as the effective ingredient of the therapeutic agent for ectopic pregnancy of the present invention, the therapeutic agent for ectopic pregnancy of the present invention may consist of the tyrosine kinase inhibitor or may be formulated into the forms suited for various administration forms using a pharmaceutically acceptable carrier(s) and/or diluent(s). Methods of formulation and various carriers therefor are well-known in the art of formulation of pharmaceuticals. The pharmaceutically acceptable carrier or diluent may be, for example, a buffer such as physiological saline or a vehicle (sucrose, lactose, corn starch, calcium phosphate, sorbitol, glycine or the like), and a binder (such as syrup, gelatin, gum arabic, sorbitol, polyvinyl chloride, tragacanth or the like), a lubricant (magnesium stearate, polyethylene glycol, talc, silica or the like) and/or the like may be appropriately admixed. Examples of the administration forms include oral formulations such as tablets, capsules, granules, powders and syrups; and parenteral formulations such as inhalants, injection solutions, suppositories and liquids. These can be prepared by generally known formulation methods.

As described above, a substance which suppresses the binding between BDNF and TrkB can also be employed as the effective ingredient of the therapeutic agent for ectopic pregnancy of the present invention. Examples of such a substance include free TrkB and TrkB fragments having an ability to bind to BDNF. Since free TrkB binds to BDNF, when free TrkB is administered, the administered TrkB competes with the original TrkB on the cell membranes and binds to BDNF. As a result, the amount of BDNF which binds to the original TrkB on the cell membranes is decreased. Thus, the free TrkB competingly suppresses the binding between the TrkB of the cells and BDNF. The site at which the TrkB on the cell membranes binds to BDNF is the ectodomain of TrkB. Therefore, as will be concretely described in the Examples below, the ectodomain of TrkB and TrkB fragments containing the ectodomain also competingly suppress the binding between BDNF and TrkB similar to the full length TrkB, so that they can be used as the effective ingredient of the therapeutic agent for ectopic pregnancy of the present invention. The base sequence of the cDNA of human TrkB gene is shown in SEQ ID NO:1 together with the amino acid sequence encoded thereby, and the amino acid sequence alone extracted therefrom is shown in SEQ ID NO:2. The cDNA of human TrkB gene and the amino acid sequenced encoded thereby are known and registered as GenBank Accession No. NM_{—}006180. In the amino acid sequence of SEQ ID NO:2 (i.e., the amino acid sequence of the full length of TrkB), the ectodomain is from −31st amino acid from the N-terminal (hereinafter referred to as “−31aa”) to 397aa. The TrkB fragment composed of this ectodomain can also be used as the effective ingredient of the therapeutic agent for ectopic pregnancy of the present invention. In general, since smaller size of the polypeptide gives easier preparation and easier intake into the cells, the above-described TrkB fragment is preferred from these points of view.

In general, it is well-known in the art that there are cases wherein the physiological activity of a physiologically active protein is retained even if the amino acid sequence of the protein is modified such that a small number of amino acids are substituted, deleted, and/or inserted. Therefore, in addition to the above-described TrkB or the fragments thereof, a polypeptide having an amino acid sequence with a sequence identity of not less than 90%, preferably not less than 95%, still more preferably not less than 99% to the amino acid sequence from −31aa to 397aa of the ectodomain in the amino acid sequence of SEQ ID NO:2, which polypeptide binds to BDNF and exerts a therapeutic effect against ectopic pregnancy can also be used as the effective ingredient of the therapeutic agent for ectopic pregnancy of the present invention similar to the free TrkB or the ectodomain fragment thereof The sequence identity of the amino acid sequence herein means a value calculated by aligning two amino acid sequences such that the number of matched amino acids is maximum (by insertion of a gap(s), as required) and dividing the number of matched amino acids by the number of amino acids of the full-length sequence (in cases where the total number of amino acids is different between the two amino acids, the number of amino acids of the longer sequence). Such calculation of the homology can be easily carried out using well-known software such as BLAST. A polypeptide having the same amino acid sequence as the amino acid sequence of SEQ ID NO:2 or the same amino acid sequence as the amino acid sequence of −31aa to 397aa in this amino acid sequence, except that one to several amino acids are substituted and/or deleted, and/or one to several amino acids are inserted and/or added, which polypeptide has an ability to bind to BDNF, and in turn, has a therapeutic effect for ectopic pregnancy, can also be used as the effective ingredient of the therapeutic agent for ectopic pregnancy of the present invention. The 20 kinds of amino acids constituting naturally occurring proteins can be classified based on the similarity of properties into neutral amino acids having a low-polar side chain (Gly, Ile, Val, Leu, Ala, Met, Pro); neutral amino acids having a hydrophilic side chain (Asn, Gln, Thr, Ser, Tyr, Cys), acidic amino acids (Asp, Glu), basic amino acids (Arg, Lys, His) and aromatic amino acids (Phe, Tyr, Trp), and it is known that substitution of amino acids within each of these classes does not alter the properties of the polypeptide in most cases. Therefore, when substituting an amino acid(s) in the polypeptide having the amino acid sequence of SEQ ID NO:2 or the ectodomain thereof, by substituting the amino acid(s) within each of these classes, the probability that the ability to bind to BDNF of the polypeptide is retained is high.

As is apparent from the fact that there are cases where a fused polypeptide constituted by ligating two types of polypeptides each having a physiological activity retains the physiological activities of the respective polypeptides, it is well-known by those skilled in the art that there are cases where a polypeptide containing an entire polypeptide having a physiological activity and an amino acid sequence(s) attached to one or two terminals thereof retains the physiological activity. Therefore, a polypeptide containing a polypeptide having an ability to bind to BDNF, which former polypeptide has an ability to bind to BDNF can also be used as the effective ingredient of the therapeutic agent for ectopic pregnancy of the present invention. In this case, although the number of the amino acids attached to one or both terminals of the above-described polypeptide having an ability to bind to BDNF is not limited as long as the resulting polypeptide has an ability to bind to BDNF, and in turn, having the therapeutic effect against ectopic pregnancy, in view of the ease of synthesis and of the activity per a unit weight, the number of the amino acids attached to one or both terminals of the polypeptide is preferably one to several.

In general, polypeptide formulations are widely used in which a polyethylene glycol (PEG) chain or the like is attached to one terminal of the polypeptide in order to make it difficult to be decomposed by proteases in the body. In the therapeutic agent for ectopic pregnancy of the present invention too, a polypeptide containing the entire polypeptide described above and a stabilizing structure such as a PEG chain attached to one terminal thereof can also be used as the effective ingredient. In cases where the peptide is stabilized by pegylation, the size in terms of molecular weight of the PEG is several thousands to 50,000, preferably about 10,000 to 50,000. The method of binding PEG to one end of a polypeptide is well-known.

In the present specification and claims, the term “modification” of the free TrkB or of the fragment thereof having the therapeutic effect against ectopic pregnancy herein means the above-described polypeptides having an amino acid sequence different from the amino acid sequence of SEQ ID NO:2 or from the amino acid sequence of the ectodomain thereof, and having the ability to bind to BDNF and in turn, having the therapeutic effect against ectopic pregnancy, as well as these polypeptides to which a stabilizing structure such as PEG chain is attached.

In cases where the above-described free TrkB, an ectodomain fragment thereof or the above-described modification (hereinafter referred to as “BDNF-binding TrkB fragment or the like” for convenience) is used as the effective ingredient of the therapeutic agent for ectopic pregnancy, the administration route may be oral route or a parenteral route. In case of a parenteral route, it can be administered via various usual administration routes such as direct administration to the site of the ectopic pregnancy, intravenous, intramuscular, subcutaneous, intracutaneous, percutaneous, rectal and instillation routes. In view of the absorption into the body and of avoiding the decomposition by digestive enzymes, parenteral administration is preferred. The dose of administration is appropriately selected depending on the type of the tyrosine kinase inhibitor, state of the patient and so on, the dose per adult per day is usually about 1 mg to 100,000 mg, preferably 1 mg to 1000 mg. However, needless to say, the dose is not restricted to this range. In cases where the BDNF-binding TrkB fragment or the like is used as the effective ingredient too, the formulation can be attained by a conventional method similarly as described above.

Although the BDNF-binding TrkB fragment or the like by itself can be used as the effective ingredient, a recombinant vector containing a nucleic acid encoding the BDNF-binding TrkB fragment or the like, which can express the BDNF-binding TrkB fragment or the like in a cell can also be used as the effective ingredient. Various vectors for gene therapy of mammals are known, and not a few of them are commercially available. Thus, a recombinant vector obtained by inserting a DNA encoding the BDNF-binding TrkB fragment or the like into the cloning site of a commercially available vector for gene therapy can preferably be employed. Fee-charging services for inserting a desired gene into a vector to construct a recombinant vector for gene therapy are available, and such a fee-charging service may also be used.

Administration itself of the recombinant vector to a mammal can be carried out by a well-known method. That is, preferably, the recombinant vector may be administered parenterally to the tissue in the vicinity of the site of ectopic pregnancy to be treated by injection or the like. A suspension obtained by suspending the recombinant vector in a buffer such as phosphate buffered saline (PBS) may be administered. To facilitate the introduction of the gene vaccine into the cells, an electric field pulse may be applied to the site of injection. In this case, the strength of the electric field is not restricted and usually about 10 V/cm to 60 V/cm, preferably about 25 V/cm to 35 V/cm, and the period of keeping the pulse is usually 20 milli seconds to 100 milli seconds, preferably about 40 milli seconds to 60 milli seconds. The pulse may be usually applied once to 6 times, preferably about twice to 4 times. Although the dose of the recombinant vector may be appropriately selected depending on the symptom and the state of the damaged site of the nerve, the dose is usually about 1 ng to 10 mg, especially about 100 ng to 1 mg in terms of the weight of the recombinant vector.

As a substance which suppresses the binding between BDNF and TrkB, an antibody to BDNF or an antibody to the BDNF-binding TrkB fragment or the like may also be used. Since BDNF and the BDNF-binding TrkB fragment or the like are readily available, antibodies to these can be obtained by a conventional method comprising administering BDNF or TrkB as an immunogen to an animal (excluding human) to induce an antibody. The antibody may be either a polyclonal antibody or a monoclonal antibody, and the monoclonal antibody can also be prepared by the conventional hybridoma method. In case of a monoclonal antibody, since it is necessary that the antibody can suppress the binding between BDNF and TrkB, a monoclonal antibody which suppresses the binding between BDNF and TrkB is screened. In case of a polyclonal antibody, since various antibodies to all of the epitopes in the immunogen are contained, an antibody which suppresses the binding between BDNF and TrkB is obtained even without carrying out such a screening.

In cases where the above-described antibody is used as the effective ingredient of the therapeutic agent for ectopic pregnancy, the administration route may be oral route or a parenteral route. In case of a parenteral route, it can be administered via various usual administration routes such as direct administration to the site of the ectopic pregnancy, intravenous, intramuscular, subcutaneous, intracutaneous, percutaneous, rectal and instillation routes. In view of the absorption into the body and of avoiding the decomposition by digestive enzymes, parenteral administration is preferred. The dose of administration is appropriately selected depending on the titer of the antibody, state of the patient and so on, the dose per adult per day is usually about 1 mg to 100,000 mg, preferably 1 mg to 1000 mg. However, needless to say, the dose is not restricted to this range. In cases where the antibody is used as the effective ingredient too, the formulation can be attained by a conventional method similarly as described above.

A substance which suppresses the production of BDNF or TrkB in the body may also be used as the effective ingredient of the therapeutic agent for ectopic pregnancy of the present invention. Examples of such a substance include interfering RNAs (iRNAs) against the BDNF gene or TrkB gene. Examples of the substance which suppresses the expression of the BDNF gene or TrkB gene include iRNAs, preferably siRNAs targeting the mRNA of the BDNF gene or TrkB gene. An iRNA is a double-stranded RNA containing a strand complementary to the target mRNA, which binds to the target mRNA and cleave it. An siRNA is a small iRNA having a size of about 21 to 23 bases. Since siRNAs have a small size, the synthesis thereof is easy and the cleavage site thereby in the mRNA can easily be selected, using an siRNA is preferred. The technique of suppressing the gene expression by an siRNA is well-known and a number of vendors providing the service to design an siRNA targeting the sequence of the mRNA (cDNA sequence) presented by a client and to construct a recombinant vector in which the siRNA is incorporated. As described above, since the sequence of the cDNA of the TrkB gene is set forth in SEQ ID NO:1, and the base sequence (GenBank Accession No. NM_{—}170735) of the cDNA of the BDNF gene is set forth in SEQ ID NO:3, siRNAs to these can be easily designed by those skilled in the art. Briefly, an siRNA is a double-stranded RNA containing a strand complementary to the mRNA to be targeted, whose size is usually 21 to 23 bases, and usually has hung over regions at both ends of the double-stranded RNA. The size of the hung over regions is 1 to 2 bases, respectively, and the hung over regions may be composed of deoxynucleotide(s). Although the complementarity to the mRNA is preferably complete, there are many cases where the siRNA has a sufficient cleaving activity even if there is a mismatch of about 1 or 2 bases. The hung over regions may not be complementary. In many cases, it is preferred to design the siRNA such that it has a sequence of aa in the sequence of mRNA and subsequent 19 to 21 bases, and one having a gc content of about 50% (usually about 45 to 55%) is preferred. Further, in order not to be cleaved during the maturation to a mature protein, in many cases, the siRNA is designed to the site apart from the 5′-end by 50 bases or more.

Although the siRNA may be administered as it is, a recombinant vector obtained by incorporating a DNA expressing the siRNA into an expression vector for mammalian cells may also be administered to produce the siRNA in the cells to suppress the expression of the BDNF gene or TrkB gene. Various expression vectors for mammalian cells are commercially available, and the above-described DNA may be inserted into the multicloning site thereof. Services by vendors who construct the expression vectors incorporating a DNA expressing an siRNA may also be used.

Although the dose of administration is appropriately selected depending on the stage of the development of ectopic pregnancy, state of the patient and so on, in cases where the suppressor is an siRNA, the dose per adult (body weight: 60 kg) per day is usually about 0.01 mg/kg to 10mg/kg, especially about 0.1 mg/kg to 5 mg/kg, and in cases where the suppressor is a recombinant vector expressing the siRNA, the dose throughout the therapy per adult per day is about 0.01 mg/kg to 10 mg/kg, especially about 0.1 mg/kg to 5 mg/kg. However, needless to say, the dose is not restricted to this range.

Further, as the effective ingredient of the therapeutic agent for ectopic pregnancy of the present invention, antisense RNAs against the BDNF gene or TrkB gene may also be employed. An antisense RNA has a base sequence complementary to the full length or a part of the mRNA of the target gene, and hybridizes with the mRNA to suppress the translation of the mRNA and in turn, to suppress the production of the gene product of the target gene. Since the base sequences of the cDNAs of TrkB gene and BDNF gene are set forth in SEQ ID NO:1 and SEQ ID NO:3, respectively, antisense RNAs to these genes can also be easily prepared. The size of the antisense RNA is not restricted as long as it can specifically hybridize with the mRNA of the target gene to suppress the translation of the mRNA, the size is usually about 20 bases to the full length of the coding region of the mRNA.

Similar to the case of iRNA, although the antisense RNA itself may also be administered, a recombinant vector obtained by incorporating a DNA expressing the antisense RNA into an expression vector for mammalian cells may be administered to produce the antisense RNA in the cells to suppress the expression of the BDNF gene or TrkB gene. Various expression vectors for mammalian cells are commercially available, and the above-described DNA may be inserted into the multicloning site thereof.

The dose of administration of the antisense RNA is appropriately selected based on the stage of the development of the ectopic pregnancy, the state of the patient and so on, and the dose may be about the same as the above-described dose of iRNA.

Based on the discovery that the suppression of the signal of BDNF/TrkB suppresses the growth of the cytotrophoblast cells and the extravillous trophoblast cells differentiated from the cytotrophoblast cells, the present invention also provides the following screening method:

That is, the present invention provides a method of screening a therapeutic agent for ectopic pregnancy, the method comprising measuring the kinase activity of TrkB in the presence of a test substance and the kinase activity of TrkB in the absence of the test substance; and selecting the test substance which decreases the kinase activity of TrkB.

Here, as the test sample, low molecular compounds, peptides, nucleic acid molecules, antibodies and the like may be employed. The kinase activity of TrkB can be measured by, for example, detecting the autophosphorylation of TrkB using an anti-phosphotyrosine antibody, although the method is not restricted thereto. Here, the kinase activity of TrkB in the cells is preferably measured.

To effectively measure the kinase activity of TrkB, various devisings may be employed. For example, the method such as that described in M. D. Sadick et al., 1997, Exp. Cell. Res., 234, 354-361 (Non-patent Document 36) may be employed. That is, TrkB fused with a peptide with 26 amino acid residues of glycoprotein D is expressed in CHO cells, and BDNF is extracellularly administered thereto to activate TrkB. The cells are then lysed and TrkB is captured on the well coated with an antibody specific to the peptide of glycoprotein D, and the autophosphorylation of TrkB is detected using a labeled anti-phosphotyrosine antibody, thereby measuring the kinase activity of TrkB.

As described above, the present invention also provides a method of screening a therapeutic agent for ectopic pregnancy, the method comprising the following steps (a) to (d):
(a) preparing model animals in which human placental villi are transplanted to a renal tissue of a mammal other than human;
(b) administering a test sample to one (or one population) of the model animals prepared and raising the animal(s), and administering only the carrier in the test sample to another (or another population) of the model animals prepared and raising the animal(s);
(c) comparing cytotrophoblast cells and extravillous trophoblast cells in the renal tissue in the model animal(s) to which the test sample was administered with cytotrophoblast cells and extravillous trophoblast cells in the renal tissue in the model animal(s) to which the test sample was not administered; and
(d) selecting the test sample as a therapeutic agent for ectopic pregnancy, which test sample decreased cytotrophoblast cells and extravillous trophoblast cells in the renal tissue in the model animal(s) to which the test sample was administered.

Here, as the mammal other than human used in the above-described step (a) is preferably a rodent, and more preferably, a severe immunodeficiency mouse which does not show rejection reaction against the placental villi originated from human. The duration of raising in the above-described step (b) is preferably about 3 to 20 days in view of carrying out the screening quickly. The carrier in the above-described step (b) is the diluent such as a solvent, binder, vehicle, drug delivery system or the like administered together with the test sample. Thus, a test in which the test substance and the carrier are administered is carried out, and, as a control, a test in which the carrier alone is administered is also carried out. For the comparison between the cytotrophoblast cells and extravillous trophoblast cells in the above-described step (c), it is preferred to use, although not restricted thereto, cytokeratin which is a marker of trophoblast cells for the identification of the both trophoblast cells, and HLA-G which is a marker of extravillous trophoblast cells. Further, for the purpose of detecting cell growth, antibodies to proteins which can be employed as an index of cell growth, such as PCNA antibody and Ki-67 antibody may also be used.

Since a surgery is usually carried out when the oviduct is ruptured in ectopic pregnancy, the ectopic pregnancy to be treated by the therapeutic agent for ectopic pregnancy of the present invention is usually unruptured ectopic pregnancy

As will be concretely shown in the Examples below, the therapeutic agent for ectopic pregnancy of the present invention suppresses the growth of the cytotrophoblast cells, thereby effectively treating ectopic pregnancy. Since the therapeutic agent for ectopic pregnancy is not an anticancer agent such as MTX, a systemic and severe side effect such as that brought about by MTX is not resulted.

The present invention will now be described more concretely by way of Examples. It should be noted that the present invention is not restricted to the following Examples.

### EXAMPLES

### Materials and Methods

### Human Villous Tissues

Human placental villi from first trimester (6-11 weeks), terminated for psychosocial reasons, were obtained by dilatation and curettage, whereas tissue samples of ectopic pregnancy were obtained from a patient at 8 weeks of pregnancy by laparoscopic surgery in Akita University Hospital (Akita, Japan). Gestational age was determined by the date of the last menstrual period and ultrasound measurement of crown-rump length. All tissue samples for in vitro and in vivo experiments were obtained from Japanese women between 18 and 30 yr of age (mean, 23±4.5 yr) after informed consent in agreement with our regional medical ethics committee.

### Human Trophoblast Villous Explant Culture

Preparation and cultivation of human villous explants of first trimester placentas were performed as described (Non-patent Document 13). Briefly, human placental villi at 6-8 weeks of gestation were aseptically dissected to remove decidual tissue and fetal membranes. Small pieces of placental villi (8 mg wet weight) were dissected under a stereomicroscope (Leica Microsystems, Tokyo, Japan). Each villous piece was put on Millicell-CM culture dish inserts (12-mm diameter) (Millipore, Bedford, Mass.) that were precoated with 200 μl of undiluted Matrigel Growth Factor Reduced (BD Bioscience pharmingen) and placed in 24-well culture plates. The villous pieces were covered with 150 μl of culture medium (DMEM/F12 without serum supplemented with 100 U/ml penicillin, 100 μg/ml streptomycin, and 0.25 μg/ml ascorbic acid, pH 7.4) (Invitrogen, Carlsbad, Calif.), whereas the bottom chamber contained 500 μl of culture medium. Villous pieces were cultured for 96 h with or without different doses of the soluble ectodomain of TrkB (R&D Systems, Minneapolis, Minn.), a neurotrophic (pan)-specific Trk receptor inhibitor, K252a (Calbiochem, La Jolla, Calif.) (Non-patent Document 14), or the inactive plasma membrane nonpermeable K252b (Calbiochem) (Non-patent Document 15) at 37° C. in 3% O₂/5% CO₂/92% N₂. Culture media were changed every 48 h and collected for measurement of glucose concentration.

Outgrowth of EVTs from the distal end of the villous tips (EVT outgrowth) and their migration into the surrounding Matrigel were monitored daily using the stereomicroscopy, and cultures in which ≧50% anchoring villous tips showed cell outgrowth were classified as EVT outgrowth positive as described (Non-patent Document 13). At the end of cultures, some villous explants including EVT outgrowth were subjected to RNA extraction for real-time RT-PCR to quantify the transcript levels of human leukocyte antigen-G (HLA-G). Glucose utilization by the villous explants was calculated from the difference between glucose concentration in fresh medium and that in the conditioned medium after 48 h of culture using an enzymatic assay (Mitsubishi BCL, Tokyo, Japan). The results were expressed as mg per 0.1 g wet weight tissue/48 h.

In some experiments, morphological changes in villous explants were evaluated by hematoxylin and eosin (H&E) staining. Furthermore, the activity of cellular proliferation was determined by immunohistochemical detection of proliferating cell nuclear antigen (PCNA) and Ki-67 antigens. To measure the progression of apoptosis, some villous explants were subjected to a quantitative caspase-3/7 enzyme assay as described (Non-patent Documents 12, 16). Apoptosis in villous explants was also assayed by detecting DNA fragmentation using in situ terminal deoxynucleotidyl transferase-mediated dUDP nick end-labeling (TUNEL) (Non-patent Document 17).

Xenotransplantation of Human Villi into SCID Mice

To explore the roles of endogenous TrkB signaling in human ectopic pregnancy, xenotransplantation of human placental villi at 7-8 weeks of gestation into SCID mice (C.B-17/Icr-scid/scidJcl) (CLEA Japan, Tokyo, Japan) was used as an in vivo model. The care and use of animals was approved by the Animal Research Committee, Akita University School of Medicine. In preparation of grafts, small pieces of the placental villi were dissected as described above and kept in ice-cold PBS until transplantation. After anesthetizing of the SCID mice at 8-11 weeks of age with tribromoethanol (14-20 mg/kg) (Sigma, St. Louis, Mo.), the left and right kidneys were sequentially exteriorized. A 0.5 mm incision was then made in each kidney capsule, and a piece of the placental villi (5 mg wet weight) was transplanted underneath the capsule using a blunt tip tweezers. Treatment was initiated in animals after 1 week of transplantation when murine vascular networks were known to appear in areas of cytotrophoblast invasion (Non-patent Document 18). Animals weighted between 19-22 g on day of treatment. Intraperitoneal (ip) administration of K252a dissolved in physiological saline (500 μg/kg) was performed daily. For negative controls, treatment with K252b (500 μg/kg) or vehicle alone was used. The doses of K252a and K252b chosen for these experiments were based on previous studies (Non-patent Documents 12, 19). Some animals were treated daily with methotrexate (ip; 1 mg/kg) (Sigma) corresponding to the therapeutic dose used for medical treatment of ectopic pregnancy (Non-patent Document 3). The mice were killed after 7 days of treatment. Kidneys with villi transplants were excised and homogenized to measure hCG-β levels and caspase-3/7 activities. To identify trophoblasts in the kidney, cytokeratin, a marker for trophoblasts (Non-patent Document 20), was detected by immunohistochemistry. In addition to H&E staining, in vivo cell proliferation and apoptosis in the excised samples were evaluated by immunostaining of PCNA and Ki-67, and the TUNEL assay, respectively.

### Statistical Analysis

Chi-square test was performed to compare the proportion of EVT positive in villous explant cultures. One-way ANOVA, followed by Fisher's protected least significant difference test, was used to evaluate other differences. Data are mean±SEM.

### RT-PCR

For conventional RT-PCR to study the expression of neurotrophins (nerve growth factor, NGF, and neurotroohin-3, NT-3) and Trk receptors (TrkA, and TrkC) in human placental villi, the primers for NGF, NT-3, TrkA, TrkC, and β-actin have been described (Non-patent Document 11). PCR reactions comprised 35 cycles of amplification with denaturation at 94° C. for 30 sec, annealing at 57° C. (TrkA), 60° C. (TrkC and β-actin), or 62° C. (NGF and NT-3) for 30 sec, and elongation at 72° C. for 30 sec. For negative controls, no mRNA was included.

### Real-Time RT-PCR

Quantitative real-time RT-PCR of TrkB and human leukocyte antigen-G (HLA-G) transcript levels in placental villi and mouse kidneys with xenotransplanted human villi was performed using a SmartCycler (Takara, Tokyo, Japan) with primers and hybridization probes for TrkB and β-actin as described (Non-patent Document 32). Primers for TrkB corresponded to the catalytic kinase domain of the receptor to avoid the amplification of truncated isoforms (Non-patent Document 33). Fragmented TrkB was specifically amplified using a reported primer (Non-patent Document 37). Validated Taqman gene expression assay was used to quantify the expression of HLA-G (Applied Biosystems, Forster City, Calif.). Data were normalized based on β-actin transcript levels.

### Immunoassays

For ELISA, placental villi were homogenized in a buffer containing 137 mM NaCl, 20 mM Tris-HCl, 1% Nonidet P40, 10% glycerol, and a protease inhibitor cocktail (Roche Applied Science, Indianapolis, Ind.) before centrifugation at 8,000×g for 5 min at 4 C. Quantification of brain-derived neurotrophic factor (BDNF) and neurotrophin-4/5 (NT-4/5) in placental villi was performed using ELISA as described (Non-patent Document 32, Non-patent Document 10). The results were normalized by protein concentrations and expressed as pg of BDNF or NT-4/5 per mg of tissues.

To localize BDNF and TrkB, BDNF and TrkB immunostaining in placental villi and villous tissues of ectopic pregnancy were performed as described (Non-patent Document 11). BDNF antigen and TrkB antigen were detected by rabbit anti-BDNF polyclonal antibody (Chemicon, Temecula, Calif.) and chicken anti-TrkB polyclonal antibody (Promega, Madison, Wis.) recognizing full length TrkB were used, respectively, after dilution to 1:100. Some slides were subjected to proliferating cell nuclear antigen (PCNA) or Ki-67 immunostaining to evaluate cellular proliferation, whereas cytokeratin and HLA-G immunostaining were performed to identify trophoblasts and extravillous trophoblasts (EVTs) in the xenotransplanted human villi, respectively. After deparaffinization and dehydration, antigen retrieval was performed by autoclave heating at 121° C. for 10 min (PCNA and Ki-67), treatment with 0.4 mg/ml proteinase K (Sigma, St. Louis, Mo.) at room temperature for 5 min (cytokeratin), or microwave heating in 10 mM citrate buffer (pH 6) for 3×3 min (HLA-G). Endogenous peroxidase activities were quenched with 0.3% hydrogen peroxidase in methanol for 30 min. After blocking with 10% BSA-Tris-buffered saline (TBS) (Sigma) for 30 min, slides were incubated with either mouse anti-PCNA monoclonal antibodies (Cell Signaling Technology, Danvers, Mass.), mouse anti-Ki-67 monoclonal antibodies (DAKO, Carpinteria, Calif.), rabbit anti-cytokeratin polyclonal antibodies (DAKO), or mouse anti-HLA-G monoclonal antibodies (Abeam, Cambridge, UK) at 1:4,000, 1:200, 1:1,000, or 1:500 dilution overnight at 4° C. After three washes in TBS, slides were incubated with biotinylated anti-mouse or anti-rabbit secondary antibodies (Invitrogen, Carlsbad, Calif.) for 30 min at room temperature. After three washes, bound antibodies were visualized using a Histostain SP kit (Invitrogen). For negative controls, the primary antibody was replaced by nonimmune mouse IgG1 or nonimmune rabbit IgG (DAKO).

The human chorionic gonadotropin (hCG)-β protein levels in the renal homogenates with transplanted villi were determined using RIA (Mitsubishi BCL, Tokyo, Japan) as described (Non-patent Document 12).

### Results

### Expression of BDNF, NT-4/5, and TrkB in Human Placental Villi During Normal and Ectopic Pregnancy

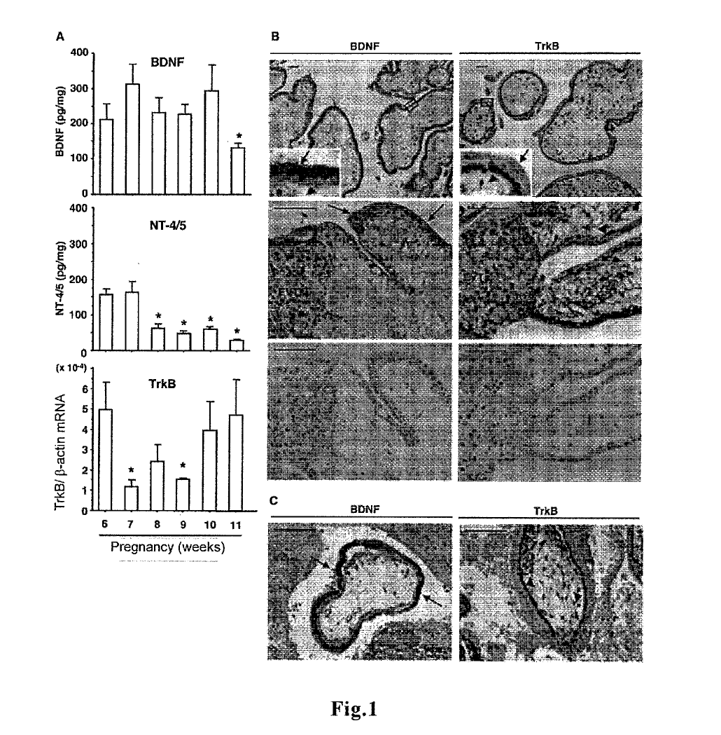

Temporal expression of BDNF, NT-4/5, and TrkB in placental villi was examined by ELISA and real-time RT-PCR during first trimester of normal gestation. In the villi, ELISA analyses indicated that BDNF protein levels were 1.3- to 5.0-fold higher than those of NT-4/5 at all the pregnant days examined (FIG. 1A). BDNF protein levels were stable during early stage but decreased at 11 weeks of pregnancy, whereas NT-4/5 protein levels were decreased after 7 weeks of pregnancy and maintained at low levels during all pregnant stages examined (FIG. 1A). In contrast, quantitative real-time RT-PCR analyses indicated that TrkB transcript levels in placental villi were high at 6 weeks, and decreased at 7 weeks of pregnancy, and gradually increased during pregnancy progression (FIG. 1A).

Cell types expressing BDNF and TrkB proteins in normal placental villi were determined by using immunohistochemistry. As shown in FIG. 1B, staining for BDNF and its receptor, TrkB were expressed in trophoblast cells of villi at 8 weeks of pregnancy in a cell type-specific manner. BDNF signal was detected in syncytiotrophoblasts and EVTs (FIG. 1B), whereas TrkB staining was localized to cytotrophoblasts and EVTs (FIG. 1B). Similar cell type-specific expressions of BDNF and TrkB proteins were detected in placental villi at 6-11 weeks of gestation (data not shown) and during ectopic pregnancy (FIG. 1C).

In vitro Inhibition of Endogenous TrkB Signaling Decreased Human Trophoblast Growth

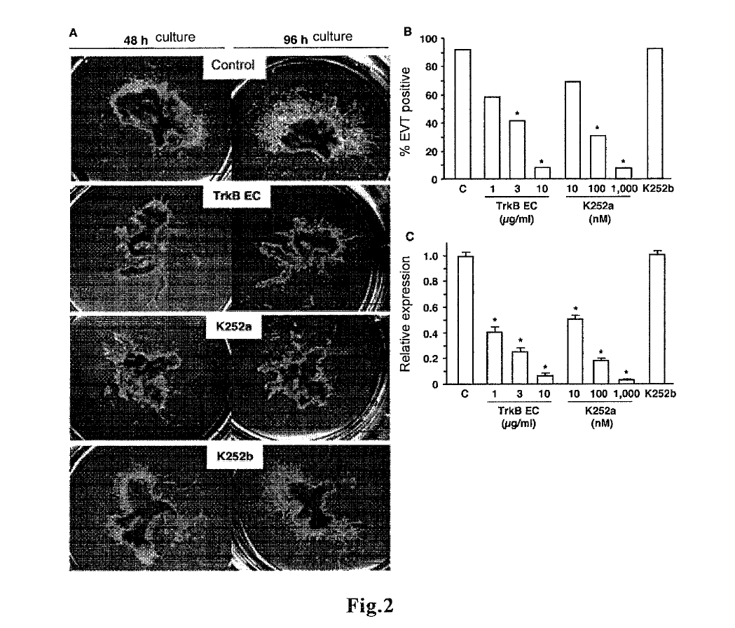

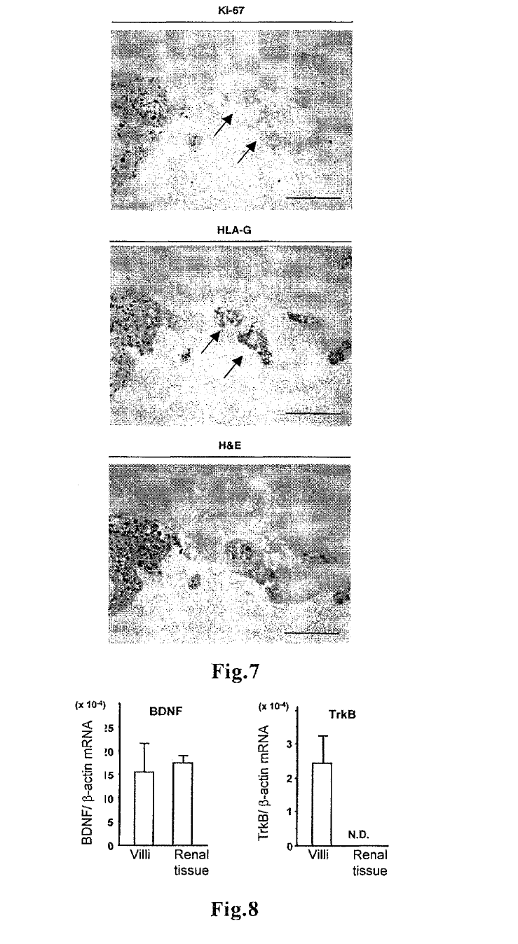

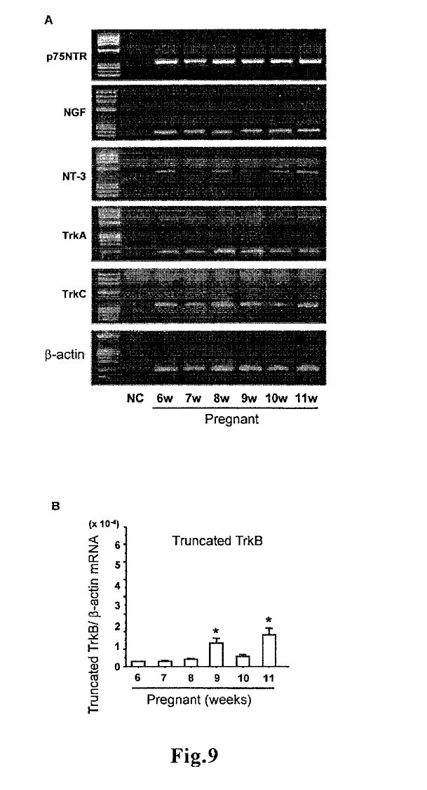

The expression of both TrkB ligands and receptors in specific cell types of human placental villi suggests that the TrkB signaling system could play an autocrine/paracrine role during trophoblast growth. To determine if endogenous TrkB ligands act as a differentiation factor for cytotrophoblasts, we evaluated EVT outgrowth from cultured villous explants treated with TrkB ectodomain and K252a. In controls, EVT outgrowth increased at 48 h of culture, and reached maximum levels at 96 h of culture accompanied with shrinkage in explant sizes (FIG. 2A). Because a cell proliferation marker (Ki-67) was not found in the migrating cells (FIG. 7), the apparent outgrowth does not involve the division of villous cells. Treatment with either the TrkB ectodomain or K252a, but not the inactive K252b, suppressed EVT outgrowth in a dose-dependent manner with similar efficacy (FIGS. 2A and B), accompanied with decreases in the transcript levels of HLA-G, a specific marker for EVT (21) (FIG. 2C).

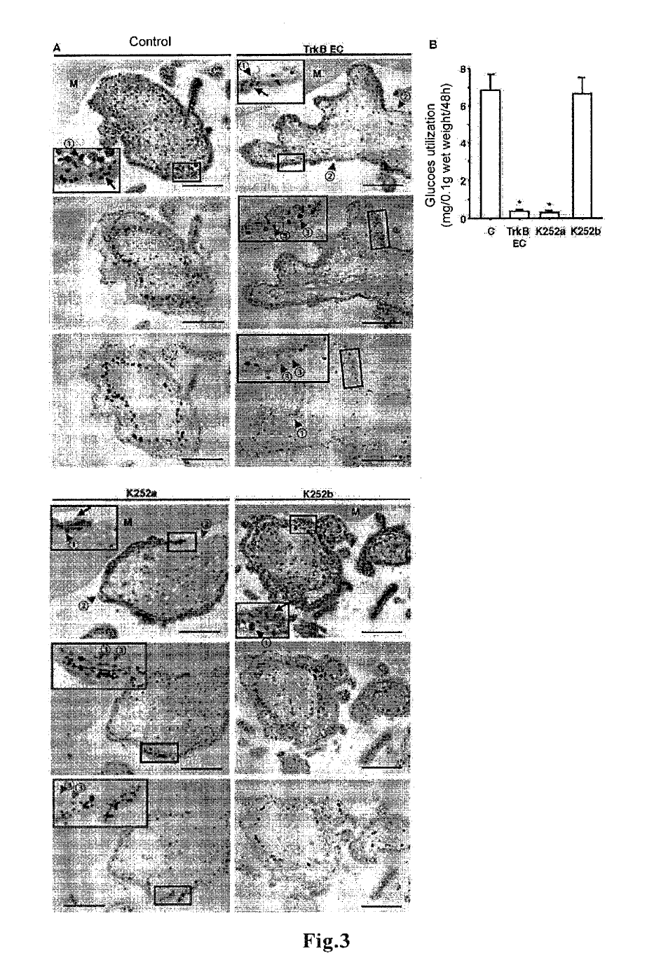

To examine effects of endogenous TrkB signaling on villous trophoblast proliferation, cellular functions were assessed morphologically and by monitoring glucose metabolism. Consistent with the expression of TrkB in specific cell types, treatment with either the TrkB ectodomain or K252a, but not the inactive K252b, decreased the number of villous cytotrophoblasts and induced partial detachment of trophoblast layers from villous stromal core at 96 h after culture (FIG. 3A, upper panels). Furthermore, we detected decreases in signals for two cell proliferation markers, PCNA (FIG. 3A, middle panels) and Ki-67 (FIG. 3A, lower panels) in remaining villous cytotrophoblasts following treatment with different inhibitors. Non-proliferative syncytiotrophoblasts were not stained with both markers in all of the control, TrkB ectodomain, K252a and K252b groups tested. Treatment with the TrkB ectodomain and K252a also decreased the glucose utilization (>94% inhibition) by villous explants (FIG. 3B), confirming reduction of their cellular viability.

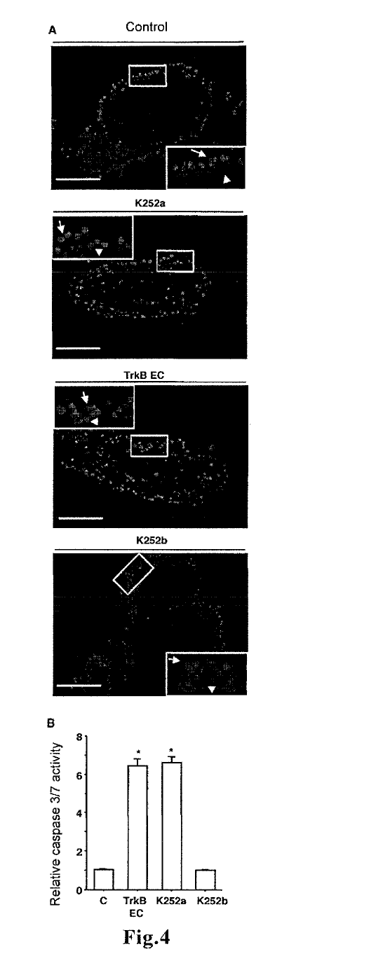

To determine if endogenous TrkB ligands act as survival factors for villous trophoblasts, we evaluated apoptosis of cultured villous explants treated with TrkB ectodomain and K252a. As shown in FIG. 4A, treatment with the TrkB ectodomain or K252a, but not the inactive K252b, increased the proportion of TUNEL-positive nuclei at 96 h after culture, thus suggesting the induction of apoptosis following suppression of endogenous TrkB ligands. The increase of TUNEL-positive nuclei was preferentially observed in cytotrophoblasts. In positive controls treated with deoxyribonuclease I, all nuclei showed TUNEL signals, whereas no TUNEL-positive nuclei were observed in negative controls (data not shown). Furthermore, we detected increases in caspase-3/7 activities by >6-fold in the villous explants following treatment with different inhibitors (FIG. 4B).

Effects of a Trk Receptor Inhibitor on Trophoblast Growth in an in vivo Animal Model of Ectopic Pregnancy

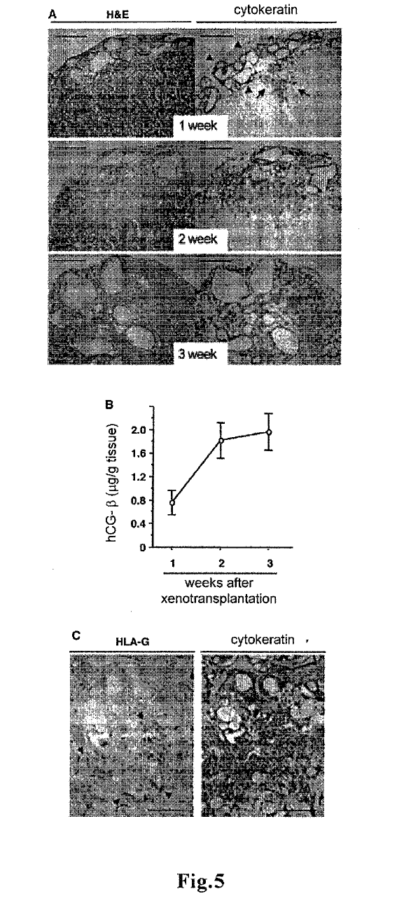

Our findings of the expression of TrkB ligands and receptors in human villi during both normal and ectopic pregnancies, and the in vitro inhibition of human trophoblast growth by TrkB inhibitors prompted us to investigate the effects of suppressing TrkB signaling as a potential treatment for ectopic pregnancy. Human placental villi were xenotransplanted into SCID mice as an in vivo model of ectopic pregnancy. Consistent with previous studies (Non-patent Document 18), trophoblast invasion into renal tissues was observed at 1 week after xenotransplantation, and the invasion was extended to deeper regions of kidney accompanied by increases in cell numbers three weeks later (FIG. 5A). Increases in hCG-β levels in tissue homogenates (FIG. 5B) further suggested the transplanted villi developed at extrauterine site. The trophoblasts invaded into kidney were stained with HLA-G (FIG. 5C), indicating their differentiation into EVTs. These findings established a model for ectopic pregnancy and allow us to test the use of Trk inhibitors for suppressing ectopic trophoblast growth.

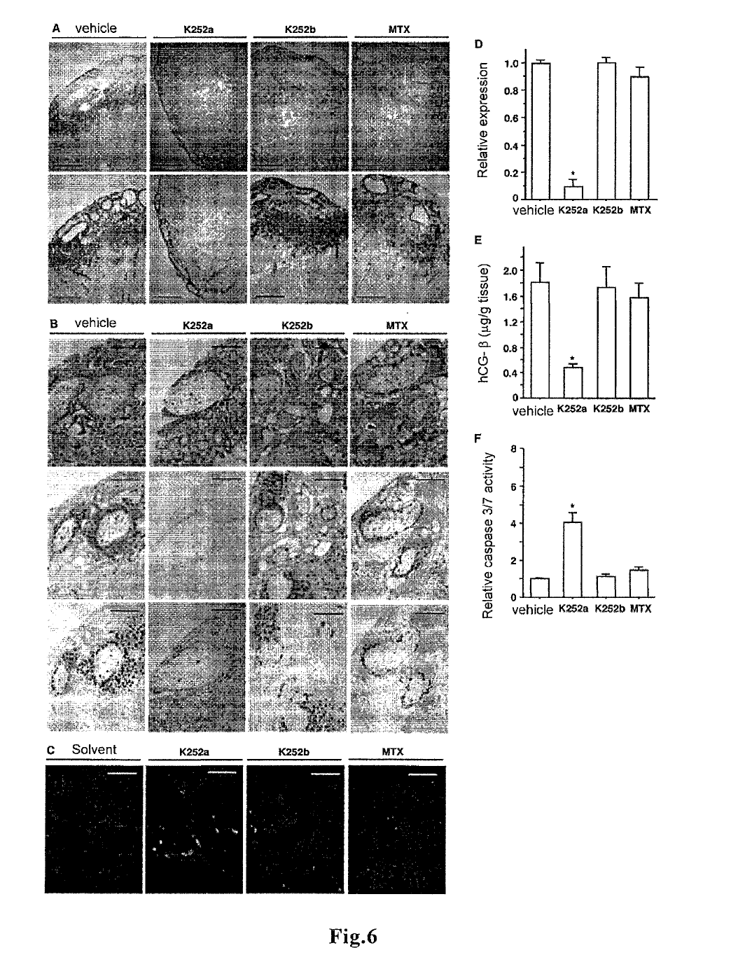

We treated mice xenotransplanted with human villi with different drugs for 7 days to evaluate their effectiveness to block ectopic pregnancy. Histopathological examination by cytokeratin, HLA-G, and H&E staining and analysis of HLA-G-expressing transcript level by real-time RT-PCR in excised kidney with transplanted villi showed decreases in cell numbers of invading EVTs and cytotrophoblasts in villous cores in the K252a group (FIGS. 6A and B). There was also a major decreases in transcript levels for HLA-G (FIG. 6D), suggesting suppression of cell differentiation and proliferation by K252a. PCNA (FIG. 6B, upper panel) and Ki-67 (FIG. 6B, middle panel) staining confirmed the effects of K252a treatment on the suppression of cell proliferation. Decreases in hCG-β levels by 73.3% in tissue homogenates following K252a treatment indicated a loss of cellular viability to synthesize hCG (FIG. 6E). This was accompanied by increases in TUNEL-positive nuclei in cytotrophoblasts after K252a treatment (FIG. 6C). We further characterized apoptosis in transplanted villi by quantifying caspases activities and observed increased activation of caspase-3/7 by 4.1-fold within the xenografts of K252a-treated mice (FIG. 6F). Of importance, the inactive plasma membrane nonpermeable K252b was ineffective for all parameters tested. Furthermore, 1 mg/kg of MTX treatment did not inhibit trophoblast differentiation, proliferation, and survival (FIG. 6A-F). Similar to our previous studies (Non-patent Document 12), no obvious side effect was observed throughout experiments in all tested animals, and no body weight changes were found in K252a-treated group during studies (vehicle, 19.62±0.95 g; K252a, 19.27±1.03 g, and K252b, 20.14±1.13 g).

1. A therapeutic agent for ectopic pregnancy, comprising as an effective ingredient a suppressor of brain-derived neurotrophic factor (BDNF) and/or of brain-derived neurotrophic factor receptor (TrkB).

2. The therapeutic agent according to claim 1, comprising as the effective ingredient at least one selected from the group consisting of a tyrosine kinase inhibitor, a fragment thereof having an ability to bind to free TrkB or BDNF, a modification thereof having a therapeutic effect for ectopic pregnancy, a recombinant vector producing said fragment or said modification in a cell, an interfering RNA against BDNF gene or TrkB gene, a recombinant vector producing said interfering RNA in a cell, an antibody to BDNF or TrkB, an antisense nucleic acid against BDNF gene or TrkB gene and a recombinant vector producing said antisense nucleic acid in a cell.

3. The therapeutic agent according to claim 2, comprising as the effective ingredient at least one selected from the group consisting of a tyrosine kinase inhibitor, free TrkB and a TrkB fragment having an ability to bind to BDNF.

4. The therapeutic agent according to claim 3, wherein said tyrosine kinase inhibitor is a compound represented by the following Formula (1): 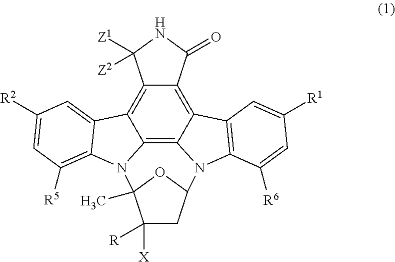
(wherein
a) both of Z¹ and Z² are hydrogen;
1) R is selected from the group consisting of OH, C₁-C₆ O-n-alkyl and C₂-C₆ O-acyl;
2) X is selected from the following group consisting of:
H;
CONHC₆H₅ with the proviso that in this case, R¹ and R² are not simultaneously Br;
CH₂Y wherein Y is OR⁷ (wherein R⁷ is H or C₂-C₅ acyl);
SOR⁸ wherein R⁸ is C₁-C₃ alkyl, aryl or a nitrogen-containing heterocyclic group;
NR⁹R¹⁰ wherein R⁹ and R¹⁰ are independently H or C₁-C₃ alkyl, Pro, Ser, Gly, Lys or C₂-C₅ acyl with the proviso that only one of R⁹ and R¹⁰ is Pro, Ser, Gly, Lys or acyl;
SR¹⁶ wherein R¹⁶ is aryl, C₁-C₃ alkyl or a nitrogen-containing heterocyclic group;
N₃;
CO₂CH₃;
S-Glc;
CONR¹¹R¹² wherein R¹¹ and R¹² are independently H, C₁-C₆ alkyl, C₆H₅ or C₁-C₆ hydroxyalkyl, or R¹¹ and R¹² together form —CH₂CH₂OCH₂CH₂—;
CH═NNHCONH₂;
CONHOH;
CH═NOH;
CH═NNHC(═NH)NH₂; 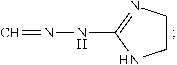
CH═NN(R¹⁷)₂ wherein R¹⁷ is aryl;
CH₂NHCONHR¹⁸ wherein R¹⁸ is lower alkyl or aryl; or
X and R together form —CH₂NHCO₂—, CH₂OH(CH₃)₂O—, ═O or —CH₂N(CH₃)CO₂;
3) R¹, R², R⁵ and R⁶ are independently H, or two or less of these are F, Cl, Br, I, NO₂, CN, OH, NHCONHR¹³, CH₂OR¹³, C₁-C₃ alkyl, CH₂OCONHR¹⁴ or NHCO₂R¹⁴, wherein R¹⁴ is lower alkyl; CH(SC₆H₅)₂ or CH(—SCH₂CH₂S—);
R¹ is CH₂S(O)ₚR²¹ and R², R⁵ and R⁶ are H wherein p is 0 or 1, R²¹ is aryl, C₁-C₃ alkyl, a nitrogen-containing heterocyclic group, 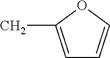
or CH₂CH₂N(CH₃)₂;
R¹ is CH═NHR²²R²³ and R², R⁵ and R⁶ are H, wherein R²² and R²³ are independently H, C₁-C₃ alkyl, C(═NH)NH₂ or a nitrogen-containing heterocyclic group, or R²² and R²³ together form —(CH₂)₄—, —(CH₂CH₂OCH₂CH₂)— or —CH₂CH₂N(CH₃)CH₂CH₂—, with the proviso that R²² and R²³ cannot be simultaneously H, and that at least one of R²² and R²³ is H except for the cases where both of these are alkyl;
(b) in cases where Z¹ and Z² together represent O, X is CO₂CH₃, R is OH, and each of R¹, R², R⁵ and R⁶ represents hydrogen).

5. The therapeutic agent according to claim 4, wherein said tyrosine kinase inhibitor is K252a.

6. The therapeutic agent according to claim 3, wherein the free TrkB or the fragment thereof having an ability to bind to BDNF is one containing ectodomain of TrkB which binds to BDNF.

7. The therapeutic agent according to any one of claims 1 to 6, wherein said ectopic pregnancy is unruptured ectopic pregnancy.

8. A method of screening a therapeutic agent for ectopic pregnancy, said method comprising measuring the kinase activity of TrkB in the presence of a test substance and the kinase activity of TrkB in the absence of said test substance; and selecting a test substance which decreases the kinase activity of TrkB.

9. A method of screening a therapeutic agent for ectopic pregnancy, said method comprising the following steps (a) to (d):
(a) preparing model animals in which human placental villi are transplanted to a renal tissue of a mammal other than human;
(b) administering a test sample to one (or one population) of said model animals prepared and raising the animal(s), and administering only the carrier in said test sample to another (or another population) of said model animals prepared and raisin the animal(s);
(c) comparing cytotrophoblast cells and extravillous trophoblast cells in said renal tissue in said model animal(s) to which said test sample was administered with cytotrophoblast cells and extravillous trophoblast cells in said renal tissue in said model animal(s) to which said test sample was not administered; and
(d) selecting the test sample as a therapeutic agent for ectopic pregnancy, which test sample decreased cytotrophoblast cells and extravillous trophoblast cells in said renal tissue in said model animal(s) to which said test sample was administered.

10. A suppressor of brain-derived neurotrophic factor (BDNF) and/or of brain-derived neurotrophic factor receptor (TrkB) for use in the treatment of ectopic pregnancy.

11. A method of treating ectopic pregnancy, said method comprising administering an effective amount of a suppressor of brain-derived neurotrophic factor (BDNF) and/or of brain-derived neurotrophic factor receptor (TrkB) to a patient with ectopic pregnancy.
